(19) United States
(12) Patent Application Publication
(10) Pub. No.: US 2025/0114121 A1
(43) Pub. Date: Apr. 10, 2025

(54) SURGICAL CANNULA WITH A LENGTH SELF-ADJUSTING MECHANISM AND MOVABLE NEGATIVE PRESSURE SEAL ANCHOR

(71) Applicants: Edward J. Mikol, Pawleys Island, SC (US); Frank Patterson, Travelers Rest, SC (US)

(72) Inventors: Edward J. Mikol, Pawleys Island, SC (US); Frank Patterson, Travelers Rest, SC (US)

(21) Appl. No.: 18/910,156

(22) Filed: Oct. 9, 2024

Related U.S. Application Data
(60) Provisional application No. 63/543,167, filed on Oct. 9, 2023.
(60) Provisional application No. 63/652,756, filed on May 29, 2024.

(51) Int. Cl. A61B 17/34; A61B 17/00

(52) U.S. Cl. CPC A61B 17/3421; A61B 2017/00367; A61B 2017/00477; A61B 2017/00561; A61B 2017/00991; A61B 2017/3437; A61B 2017/3445; A61B 2217/005

(57) ABSTRACT

Disclosed are embodiments of a retractable surgical cannula with a self-adjusted mechanism to shorten the length or the effective length of the cannula to the thickness of the cut tissue after insertion into a patient's body or cavity. The self-adjusted mechanism may comprise a telescopic cannula body, or may comprise a sliding collar on a cannula body which may be telescopic or non-telescopic. Also disclosed is a vacuum seal mechanism to seal the cannula to the patient's skin and to evacuate fluids from the cavity that may leak around the cannula. The vacuum seal mechanism, which can be the sliding collar provided with a suctioning force, may be movable relative to the cannula body.

### CROSS-REFERENCE TO RELATED PATENT APPLICATIONS

The present invention claims priority under 35 U.S.C. § 119 (e) to U.S. provisional patent application No. 63/543,167 filed on 9 Oct. 2023 and to U.S. provisional application No. 63/652,756 filed on 29 May 2024. Both parent provisional patent applications are incorporated herein by reference in their entireties.

### FIELDS OF THE INVENTION

The present invention is directed to a surgical cannula that can self-adjust its length from the proximal end to the distal end after insertion or deployment into the patient's body or cavity. Preferably, the surgical cannula has a telescopic body.

The present invention is also directed to a surgical cannula with a negative pressure seal that can evacuate bodily fluids that may leak from the cavity around the cannula.

The present invention is also directed to a surgical cannula that has a negative pressure seal that when activated can anchor the cannula to the patient's skin around the cut tissue or around the incision site.

The present invention is also directed to said negative pressure seal adapted to be attached to the cannula.

The present invention is further directed to a surgical length-adjusting cannula with a movable negative pressure seal that can effectively set the operating length of the cannula as between a distal anchor and the movable negative pressure seal. This surgical cannula may or may not utilize a telescopic cannula body, and may or may not have its physical length adjusted.

The present invention is further directed to a surgical cannula with a distal seal and a proximal seal in the lumen, and a distal anchor. Optionally, the surgical cannula also has a movable negative pressure seal to effectively set the operating length of the cannula.

The present invention is further directed to a method for minimizing a leakage of fluid from a patient's cavity during a surgical procedure utilizing a cannula.

### BACKGROUND OF THE INVENTION

Cannulas have been used in minimally invasive surgical procedures, such as laparoscopic and arthroscopic surgeries. During these procedures, a small incision or cut is made in the skin and tissue of a patient to access internal cavities, such as the abdomen or joints. A cannula is inserted into the cut tissue and is secured to the incision site. Surgical instruments are passed through the proximal opening of cannulas to enter a body cavity. During these procedures, the body cavity is inflated with an insufflated gas or liquid to create a surgical zone in the body cavity for surgical instruments. These cannulas generally have internal sealing members to seal the cannula's lumen during the procedures.

Telescopic cannulas having an inner sleeve slidingly disposed within an outer sleeve are generally known. U.S. Pat. No. 10,245,070 and related patents disclose various embodiments of telescopic cannulas having mechanisms that adjust the length of the cannula after insertion into the patient's body. Some mechanisms require manual adjustment of the cannula's length and others include pretensioned biased elements. It is known that pre-tensioning a biased element can over time lose its springiness or its ability to exert force once the biased element is released.

U.S. published patent application No 2023/0023504 discloses another telescopic cannula that has a manually length adjusted mechanism and a biased position retention collar to lock the length of the cannula after manual adjustments. The retention collar disposed around the cannula is biased toward the distal direction, and is held in place by the collar pressing a plurality of ball bearings into circular grooves on the outer surface of the inner member of the telescopic cannula.

The art does not disclose a mechanism to retain the cannula, preferably a proximal end or proximal flange of the cannula, to the patient's skin surrounding the cut tissue or surrounding the incision site.

There remains a need for a cannula with improved length adjustment mechanism that can automatically adjust the length of the cannula to the depth of the cut tissue. There also remains a need for a mechanism on a cannula that retains, preferably by suction, a portion of the cannula to the patient's skin surrounding the cut tissue or surrounding the incision site. There also remains a need for surgical cannula that can evacuate bodily fluids that may leak from the body cavity around the cannula and that can mitigate or minimize the same leakage.

### SUMMARY OF THE INVENTION

In one embodiment, the invention is directed to a telescoping cannula that self-adjusts its deployed length to the tissue depth and that comprises a retainer to minimize pull-out or undesirable cannula movements during surgery. The inventive cannula also comprises a suction or vacuum device that seals the cannula to the patient's skin and evacuates fluid from the body cavity that may leak around the cannula and adjusts its length as the tissues swell during the surgical procedure.

As used herein, negative pressure or vacuum is a pressure below atmospheric pressure. In one nonlimiting example, negative pressure can be about 400 mm of Hg or about 7.7 psi.

An embodiment of the present invention is a cannula comprising an outer sleeve, an inner sleeve disposable within the outer sleeve, wherein the outer sleeve and the inner sleeve are movable relative to each other to change an overall length of the cannula, a biasing element disposed and held within a space defined between the inner sleeve and the outer sleeve, and a distal anchor. The cannula is movable from a first configuration, where the biasing element is in a substantially unstretched or uncompressed state, to a second configuration, where the biasing element is compressed or stretched before being inserted into a patient's body. The overall length of the cannula is self-adjusted after insertion of the cannula into the patient's body.

Preferably, a move to the second configuration is by action of a trocar and wherein after the removal of the trocar from the cannula the distal anchor is held by an inner surface of a patient's body.

Alternatively, a move to the second configuration is by a distal movement of the inner sleeve and a relative rotation between the inner sleeve and the other sleeve, wherein after a reverse rotation between the inner sleeve and the outer sleeve the distal anchor is held by an inner surface of a patient's body. Preferably, a key is inserted into the cannula to affect said reverse rotation.

Preferably, the biasing element comprises a spring, more preferably a helical spring. Alternatively, the biasing element comprises a compressible rubber sleeve, a compressible foam sleeve or a gas-filled bladder.

In another embodiment, the inventive cannula further comprises a suctioning device comprising at least one skin contacting channel fluidly connected to a suction source, wherein the at least one skin contacting channel provides a suction force to retain the cannula to a patient's skin. Preferably, the suctioning device is positioned below a proximal flange on the cannula.

The inventive cannula preferably comprises a flex section allowing a proximal section of the cannula to flex about its centerline.

Preferably, the suctioning device is movable relative to the cannula body, and is movable along the cannula body.

Preferably, the at least one skin contacting channel of the suctioning device comprises a first skin contacting channel to retain the cannula to the patient's skin, and a second skin contacting channel to evacuate fluid leaked around the cannula. Preferably, the first and second skin contacting channels are concentric and are connected said suction source.

Alternatively, the at least one skin contacting channel of the suctioning device is connected to a porous, open-cell sleeve on the cannula.

In another embodiment of the present invention, the suctioning device adapted to be attached to a cannula, said suctioning device comprises at least one skin contacting channel fluidly connected to a suction source, wherein the at least one skin contacting channel provides a suction force to retain the cannula to a patient's skin. The suctioning device is movable relative to the cannula body or is movable along the cannula body. The suctioning device has an optional distal anchor. Preferably, the suctioning device may evacuate fluid that may leak along the length of the cannula from the cavity. Preferably, the suctioning device may retain the cannula to the patient's skin and evacuate leaked fluid.

Preferably, the suctioning device is positioned below a proximal flange on the cannula.

The suctioning device may comprise a flex section allowing a proximal section of the cannula to flex about its centerline.

Preferably, the at least one skin contacting channel comprises two concentric skin contacting channels. Preferably, the two concentric skin contacting channels are connected said suction source.

The suctioning device may be slidable along the cannula body.
a. Preferably, the suctioning device is connected to the cannula body through a bayonet type connection.
b. Preferably, the suctioning device is connected to the cannula body by a frictional seal.
c. Preferably, the suctioning device comprises a squeezable locking device connected to the frictional seal, wherein in a locking configuration the squeezable locking device is in a relaxed state and applies a pressure on the cannula body to resist a movement of the suctioning device along the cannula body, and wherein in a release configuration the squeezable locking device is elastically deformed to allow the movement of the suctioning device along the cannula body. Preferably, in the locking configuration the squeezable locking device has an oval shape with first portions on its minor axis applying pressure on the cannula body, and in the release configuration second portions on its major axis are elastically deformed to release the first portions.
d. Alternatively, the suctioning device is connected to the cannula body by a frictional seal and is biased toward the patient's skin by a spring, and the cannula is retained between the biased suctioning device and the distal anchor. In this alternative, the suctioning may be omitted.
e. Preferably, the cannula body comprises a plurality of circumferential ridges on its outer surface and the suctioning device comprises at least one inner ridge, wherein the at least one inner ridge is received between adjacent circumferential ridges.
f. Preferably, the cannula body comprises a plurality of circumferential ridges on its outer surface and the suctioning device comprises a biased actuator comprising at least one locking edge, wherein in a locking configuration the at least one locking edge is received between adjacent circumferential ridges and wherein in a release configuration the at least one locking edge is moved away from the plurality of circumferential ridges. The suctioning device can be moved from the locking configuration to the release configuration by pushing an actuating element on the biased actuator.

The suctioning device may be rotatable along the outside surface of the cannula body. Preferably, the suctioning device is connected to the cannula body through threads. The suctioning device can be positioned at an adjustable position along the length of the cannula body.

The cannula body may have a telescopic body of a single-piece body.

Still another embodiment of the present invention is a cannula comprising a casing defining a lumen sized and dimensioned to receive one or more medical instruments, an inflatable outer membrane attached to an outer surface of the casing, and a cap sized and dimensioned to move relative to the casing. At least one port formed on the casing, wherein the at least one port is fluidly connecting the outer membrane to the lumen, wherein the outer membrane is filled with a fluid and the cap is moved in a distal direction to fluidically isolate said at least one port from the outer membrane to further pressurize the outer membrane and wherein the cap comprises a suctioning device adapted to be attached to the cannula, said suctioning device comprises at least one skin contacting channel fluidly connected to a suction source, wherein the at least one skin contacting channel provides a suction force.

Preferably the cap comprises a second port and wherein the second port and said at least one port are aligned to fluidly connect the outer membrane to the lumen. Preferably, the cap is threadedly connected to the casing so that relative rotation between the cap and the casing allows the cap and the casing to move relative to each other. Preferably, the at least one skin contacting channel provides the suction force to retain the cannula to the patient's skin. Preferably, the at least one skin contacting channel provides a suction force to evacuate fluid leaked around the cannula.

Yet another embodiment of the present invention is a cannula comprising a cannula body defining a lumen sized and dimensioned for medical instrument to pass through, a proximal seal, preferably a slitted diaphragm, positioned at a proximal end of the cannula body, wherein the medical instrument can pass through the proximal seal, a distal seal, preferably another slitted diaphragm, positioned at a distal end of the cannula body, wherein the medical instrument can pass through the distal seal, wherein the proximal seal and the distal seal are spaced apart, and a distal anchor, preferably an inverted umbrella, disposed on a distal end of the cannula body.

Preferably, the distal seal and the distal anchor are integral with each other. Preferably, the distal seal and the distal anchor are threaded onto or pressure fitted to the distal end of the cannula body.

Preferably, the cannula further comprises a suctioning device adapted to be attached to an outside surface of the cannula body, said suctioning device comprises at least one skin contacting channel fluidly connected to a suction source, wherein the at least one skin contacting channel provides a suction force to retain the cannula to a patient's skin or to evacuate leaked fluid. Preferably. the suctioning device is movable relative to the cannula body.

Another embodiment of the present invention is a method for minimizing a leakage of fluid from a patient's cavity during a surgical procedure utilizing a cannula comprising the steps of:
a. inserting the cannula into the patient's cavity, wherein the cannula comprises a suctioning device, said suctioning device comprises at least one skin contacting channel;
b. fluidly connecting a suction force to the at least one skin contacting channel, said suction source provides a suction force to retain the cannula to the patient's skin; and/or
c. evacuating said fluid from the at least one skin contacting channel away from the patient's skin.

Said inventive method may further comprise the step of
d. sealing an outside surface of the cannula to a cut tissue where the cannula is inserted by an outer seal positioned on the outer surface of the cannula.

Said inventive method may further comprise the step of
e. sealing a lumen of the cannula by a lumen seal positioned proximate to a distal end of the cannula.

Any and all embodiments of the inventive cannula may comprise an outer seal positioned on the outer surface of the outer sleeve of the cannula.

Any and all embodiments of the inventive cannula may comprise a lumen seal positioned proximate to a distal end of the inner sleeve of the cannula.

As used herein, cut tissues are the tissues cut by the surgeon during the operation to insert the cannula/trocar, and do not include the patient's skin surrounding the cut tissue and do not include the tissues untouched by the surgeon's scalpel surrounding the cut tissues.

As used herein, skin or patient's skin includes a surgical film, preferably a self-adherent, breathable surgical film, that may cover the patient's skin. The suctioning collar or other suctioning/vacuuming devices described herein may apply a suction force to the patient's skin or to the surgical film covering the patient's skin.

### BRIEF DESCRIPTION OF THE DRAWINGS

In the accompanying drawings, which form a part of the specification and are to be read in conjunction therewith and in which like reference numerals are used to indicate like parts in the various views and in the various embodiments. Part(s) with the same reference numbers in the drawings and in the description are the same part, unless indicated otherwise.

FIG. 1 (a) is a front perspective view of the inventive telescopic cannula and trocar;

FIG. 1 (b) is a front view of the cannula and trocar of FIG. 1 (a).

FIG. 2 is an exploded view of the cannula and trocar of FIGS. 1 (a) and 1 (b).

FIG. 3 is a cross-sectional view of the cannula in a first relaxed configuration without the trocar.

FIG. 4 is a cross-sectional view of the cannula in a second compressed configuration with the trocar inserted therein, and the cannula and trocar are inserted into the patient's body.

FIG. 5 is a cross-sectional view of the cannula in the deployed configuration in the patient's body with the trocar withdrawn.

FIGS. 6 (a)-6 (b) are cross-sectional views of FIG. 1 (b) bisecting each of the vacuum tubes; FIG. 6 (c) is a bottom view of the suction collar; FIG. 6 (d) is a schematic representation of the inventive cannula deployed at angle from the patient's skin; and FIG. 6 (e) is a partial cross-sectional view of another cannula that can be used with the suction collar or cap.

FIG. 7 is a cross-sectional view of another embodiment of the inventive cannula.

FIG. 8 is an exploded view of another embodiment of the inventive cannula.

FIG. 9 (a) is a front perspective view of another embodiment of the present invention; FIG. 9 (b) is a schematic simplified version of FIG. 9 (a) with certain details omitted for clarity; FIG. 9 (c) is a schematic drawing of yet another embodiment of the present invention; FIGS. 9 (d) and 9 (e) are front perspective and cross-sectional views of another embodiment of the present invention.

FIGS. 10 (a) and (b) are partial cross-sectional views of another embodiment of the present invention.

FIGS. 11 (a) and (b) are cross-sectional views of a portion of FIGS. 10 (a)-(b) showing an alternative thereof.

FIGS. 12 (a) and (b) are partial cross-sectional views showing an alternative to the cannula of FIGS. 10 (a) and (b) and 11 (a) and (b).

FIGS. 13 (a) and (b) are partial cross-sectional views showing another alternative to the cannula in FIGS. 10 (a) and (b), FIGS. 11 (a) and (b) and FIGS. 12 (a) and (b).

FIG. 14 (a) shows a variation of the cannula of FIGS. 9 (a) and (b); and FIG. 14 (b) is an exploded view of FIG. 14 (a).

FIG. 15 (a) shows a variation of the cannula of FIGS. 14 (a) and (b); and FIG. 15 (b) is an exploded view of FIG. 15 (a).

FIG. 16 (a) shows a variation of the cannula of FIG. 9 (c); and FIG. 16 (b) is an exploded view of FIG. 16 (a); FIG. 16 (c) is a top view of the deformable friction seal and the suction collar in a locking configuration and FIG. 16 (d) shows the deformable friction seal in a moving configuration; FIG. 16 (e) is a cross-sectional view of the deformable friction seal along line A-A in FIG. 16 (f); FIG. 16 (g) is an alternative embodiment of FIGS. 16 (a) and (b); and FIGS. 16 (h)-16 (i) show another embodiment of FIG. 16 (g).

FIG. 17 (a) shows a variation of the cannula of FIGS. 15 (a) and (b); FIG. 17 (b) is an exploded view of FIG. 17 (a); FIG. 17 (c) is an exploded view of an assembly of the suction collar; and FIGS. 17 (d) and (e) show the suction collar assembly in a locking configuration and a release configuration.

FIGS. 18 (a)-(j) are photographs of the experiments and examples from the Appendix.

### DETAILED DESCRIPTION OF THE PREFERRED EMBODIMENTS

The invention is directed to improved surgical cannulas including and not limited to cannulas that overcome the prior art drawbacks identified above.

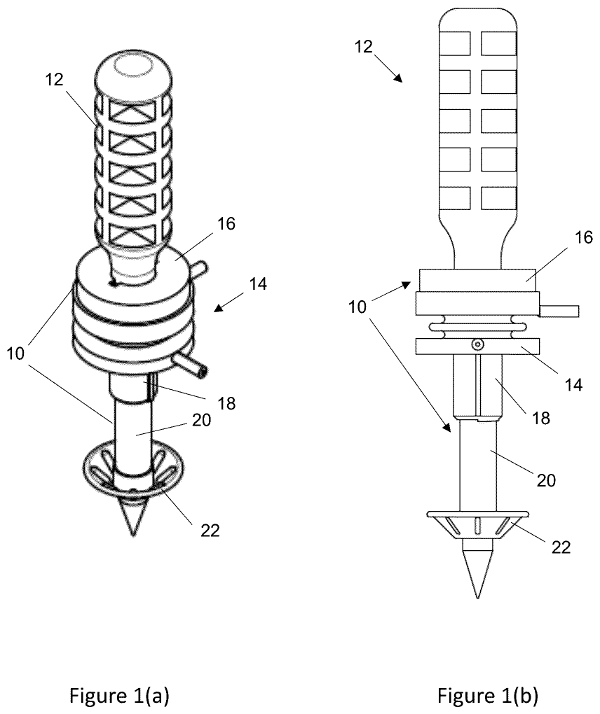

Referring to FIGS. 1 (a) and 1 (b), inventive cannula 10 and trocar 12 inserted therein are shown. Suction collar 14 is attached to a proximal flange 16 of cannula 10, as described below. In one embodiment, cannula 10 is a telescopic cannula comprising outer or proximal sleeve 18 and inner or distal sleeve 20. The inner and outer sleeves 18 and 20 are sized and dimensioned to slide, preferably freely or with only minor resistance, relative to each other. A distal anchor or distal retainer 22, which may have several embodiments, is provided at the distal end of cannula 10, preferably at the distal end of inner sleeve 20, to anchor or retain the cannula within the patient's body or cavity. Advantageously, the distal retainer 22 in the embodiments is a distal inflatable balloon or a distal umbrella, discussed below, has the ability to keep bodily fluid in the cavity from entering the space between the cut tissue and the outer surface of the cannula to minimize leakage from the cavity.

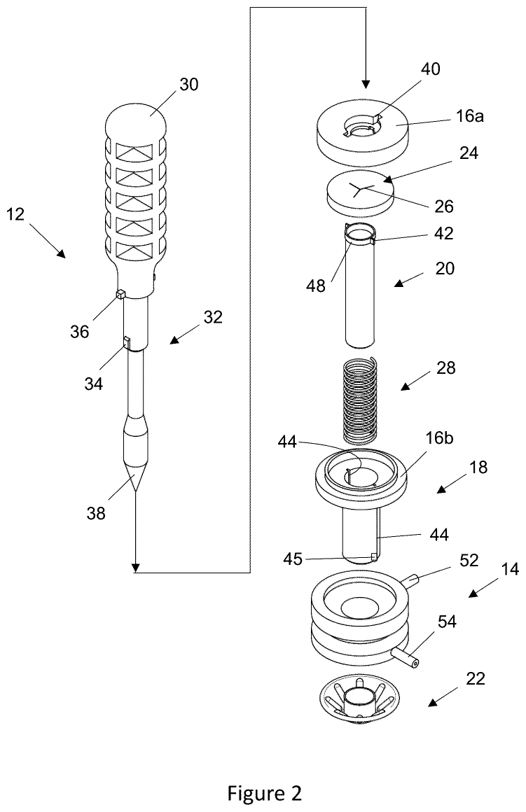

The components of cannula 10 and trocar 12 are shown in the exploded view of FIG. 2. Internal to cannula 10 are gasket 24 with slit(s) 26 and biasing element 28, preferably a helical spring, a compressible rubber sleeve or a compressible foam sleeve or a gas-filled bladder. Trocar 12 comprises a handle portion 30 sized and dimensioned for a surgeon to handle and manipulate the cannula, and insertion portion 32 to be inserted into cannula 10 to aid in the placement of cannula 10 inside the patient and to adjust the length of cannula 10. Trocar also has at least one pushing shoulder 34 adapted to push inner sleeve 20 away from outer sleeve 18, and at least one aligning shoulder 36 sized and dimensioned to pass through proximal flange 16 at a certain orientation or alignment. Distal tip 38 of insertion portion 32 is preferably narrow or pointed in the distal direction to facilitate the trocar's movement within cannula 10 and through the capsular tissue. Insertion portion 32 as shown in the drawings have various non-limiting, exemplary cross-sections. Insertion portion 32 may have any shape or profile, so long as it can move freely within cannula 10.

Proximal flange 16 is positioned on the proximal end of cannula 10, preferably on outer sleeve 18, and preferably comprises a top flange portion 16a and bottom flange portion 16b, which is preferably attached to outer sleeve 18. Top and bottom flange portions 16a and 16b are designed to fit together, preferably in a fluid tight manner, by snapping, matching threads, or other known interlocking mechanisms. Alternatively, top and bottom flange portions 16a and 16b can be molded or printed as a unitary piece. Slitted gasket 24 is disposed between the two flange portions to provide a fluid tight environment within cannula 10. Slit(s) 26 in gasket 24 is provided to allow the insertion and withdrawal of trocar 12 and medical instruments during medical operations where cannula(s) 10 are deployed. Top flange portion has indent(s) 40 that is sized and dimensioned to receive aligning shoulder(s) 36 of trocar 12 when ident(s) 40 and aligning shoulder(s) 36 are aligned with each other. Preferably, indent(s) 40 limits the travel of aligning shoulder(s) 36. Trocar 12 can be twisted or rotated to lock it under cap 16a, holding the cannula at its maximum length.

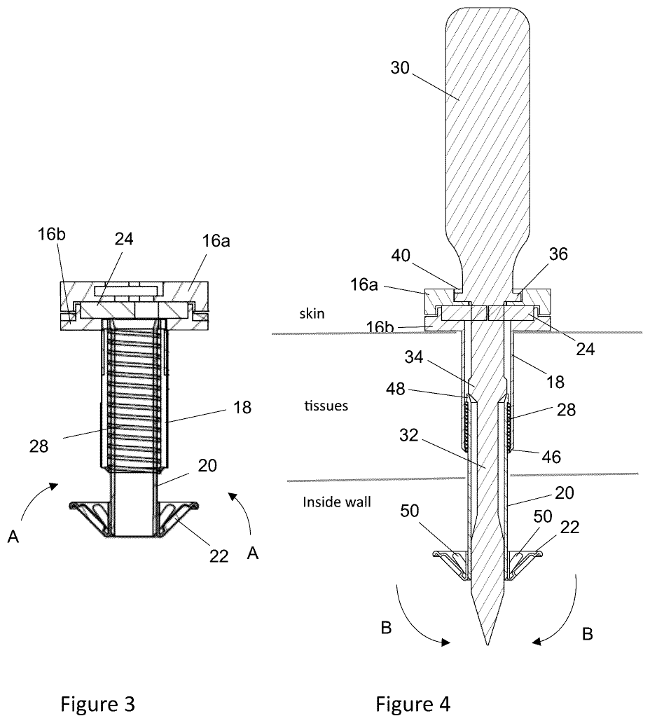

Inner sleeve 20 comprise at least one barb 42 adapted to be received and slide within at least one channel 44 on outer sleeve 18 to facilitate the relative movements between inner and outer sleeves 18 and 20. When assembled, as shown in FIGS. 3 and 4, inner and outer sleeves form a cavity therebetween that is sized and dimensioned for spring 28 to fit therein. The distal end of outer sleeve 18 is tapered inward to provide ledge 46 to hold spring 28 preventing it from slipping outside of cannula 10. Likewise, ledge 48 shown in FIG. 2 on the outside inner of sleeve 20 is positioned at the proximal end of spring 28, when cannula 10 is assembled. The cavity formed in the space between outer and inner sleeves 18 and 20 and between ledges 46 and 48 houses spring 28 to protect the spring from being contacted by surgical instruments. In other words, spring 28 does not form a part of the lumen of cannula 10. This space may also be fluidically sealed to prevent liquids from entering the cavity by the use of O-rings connected to ledges 46 and 48. Optionally, ledge 48 is sized and dimensioned to be sufficiently large so that it would not slide past ledge 46 thereby minimizing the possibility that cannula 10 separates if too much traction is applied or if distal retainer 22 is hung up.

FIG. 3 shows cannula 10 in the first or relaxed configuration without trocar 12. Preferably, cannula 10 is stored in the relaxed configuration prior to being used in a medical operation. Spring 28 is preferably in a relaxed state, i.e., un-compressed and un-extended although a small amount of compression or extension, e.g., up to ±5%, preferably ±2.5% of spring 28's undisturbed length to account for incidental compression or extension, is allowed. It is known in the art that prolonged periods of compression or extension could permanently or plastically deform the spring thereby adversely affecting the spring's ability to return to its free or undisturbed state. When this occurs, the spring loses at least some of its spring force.

FIG. 4 shows cannula 10 in the second or compressed configuration when trocar 12 is inserted into the lumen of telescopic cannula 10, through slit gasket 24. Aligning shoulder 36 lines up with indent 40 on top flange 16a, which limits how deep trocar 12 may enter cannula 10, and shoulder 36 can be twisted to lock trocar 12 in place. Pushing shoulder(s) 34 on trocar 12 contacts ledge 48 and pushes inner sleeve 20 in the distal direction thereby compressing spring 28 to store energy in the spring. As shown in FIG. 4, trocar 12 is at its maximum insertion position and inner sleeve 20 and outer sleeve 18 are at their longest combined length. Anchor 22, which in this embodiment is in the shape of an inverted umbrella, has a plurality of resilient ribs 50. Ribs 50 are pressed toward inner sleeve 20 and are folded along inner sleeve 20 in direction A during the insertion. After the insertion and through the patient's skin and cut tissues, anchor 22 re-expands in the cavity and is deployed to a diameter that is larger than that of cannula 10 and that of the cut tissue, and is sufficiently large to minimize unwanted retraction of the cannula. In other embodiments, distal retainer 22 can be a bulge of prominence of larger diameter instead of an umbrella, or the distal retainer 22 can be constructed without ribs 50.

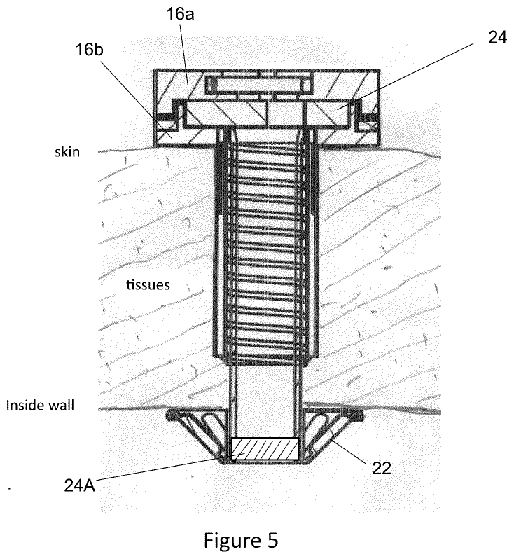

Thereafter, trocar 12 is withdrawn to clear the cannula's lumen for medical instruments. The spring energy stored in biasing element or spring 28 during the insertion is released pulling inner sleeve 20 in the proximal direction until extended umbrella anchor 22 contacts the inside wall or an inner surface surrounding the patient's cut tissue in the deployed configuration, as shown in FIG. 5. The post-insertion length of cannula is 10 self-adjusted by the action of expanding spring 28. Cannula 10 is held at the patient's skin by bottom flange 16b and by extended anchor 22 within the patient's body or cavity. Advantageously, the post-insertion length of cannula 10 is the same or substantially the same as the thickness of the patient's tissue that was cut to inert cannula 10. As illustrated in FIGS. 3, 4 and 5, the post-insertion length in FIG. 5 is generally longer than the pre-insertion length in FIG. 3 and shorter than the length during insertion in FIG. 4. The post-insertion length may also be substantially the same as either the pre-insertion length or the length during insertion, depending on the thickness of the cut tissue and the size/length of the cannula.

In one alternative, umbrella anchor 22 is stored within outer sleeve 18 in the relaxed configuration of cannula 10 and is extended when it is pushed past outer sleeve 18. In another alternative, umbrella anchor 22 is located distally from outer sleeve 18 and ribs 50 are in the extended state, as shown in FIG. 3 in the relaxed configuration. Ribs 50 is readily bent inward toward the cannula's centerline in direction A during the insertion and re-extended when passed the patient's tissue.

In another alternative, cannula 10 may be initially inserted into the cut tissue in the relaxed configuration shown in FIG. 3 and the insertion of trocar 12 through cannula 10 completes the insertion procedure. In another alternative, trocar 12 is inserted into cannula 10 before both are inserted into the patient's cut tissue.

During the withdrawal of cannula 10, extended ribs 50 are designed with the requisite resilient and flexibility so that they flipped in direction B into an inverted configuration by the withdrawing force, like an umbrella being inverted by strong winds in a storm, as demonstrated in Experiment 3 in the Appendix.

In one alternative, anchor 22 could be a self-inflating balloon or membrane. Such self-inflation could be accomplished by storing a compressible gas, e.g., air or nitrogen, in the balloon. During the insertion procedure, the gas is compressed either by being inside outer sleeve 18 or being inserted into the cut tissue. After anchor 22 is pushed past the patient's cut tissue, the balloon reinflates to anchor the cannula. During the withdrawal, the inflated gas is re-compressed by the withdrawing force allowing the cannula to be withdrawn. In another alternative, anchor 22 is a bulge or prominence of larger diameter than inner sleeve 20, which requires a higher force to insert and to withdraw.

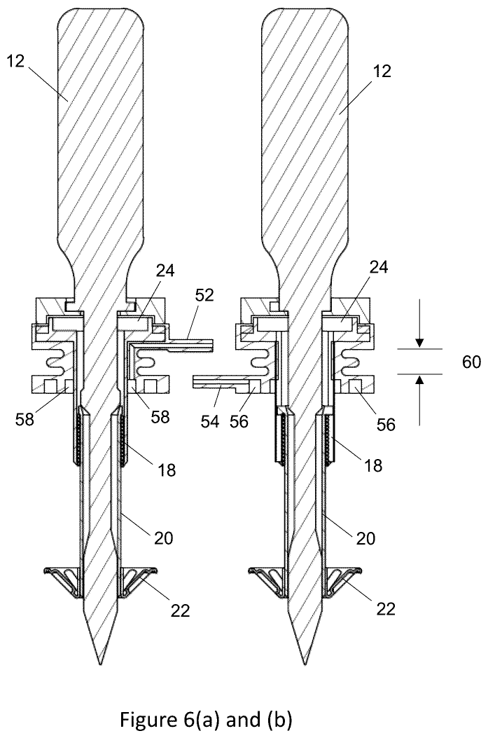

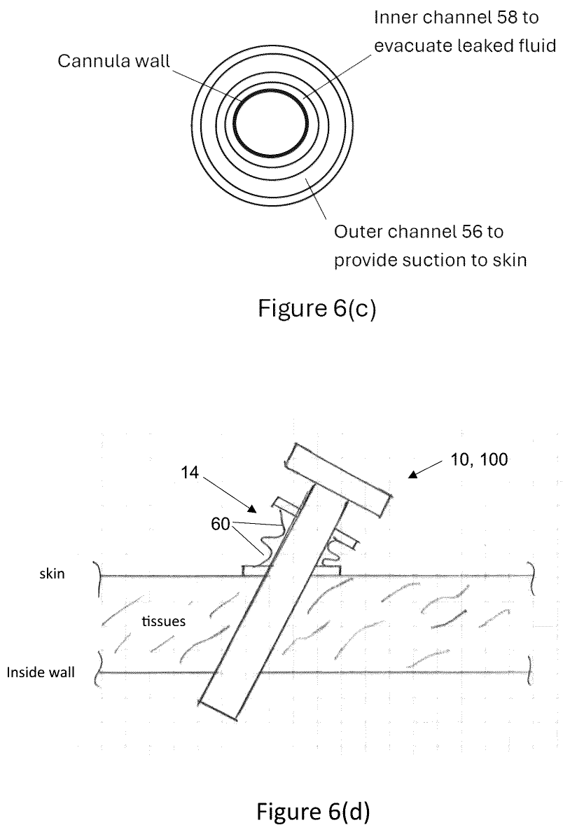

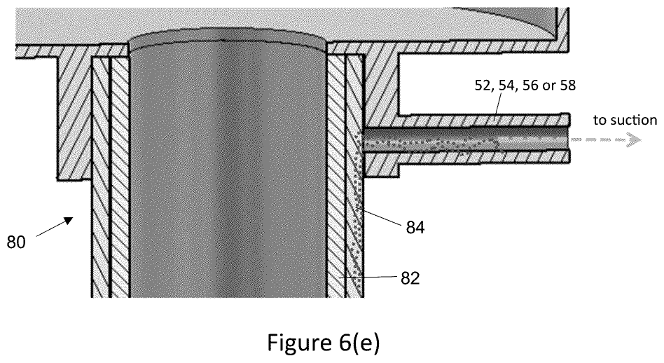

Suction collar 14 is shown in FIGS. 1 (a), 1 (b) and 2. Suction collar 14 has at least one suction connector 52, 54 and at least one skin contacting channel 56, 58, which is fluidly connected to the suction connector 52, 54 and is in contact with the patient's skin, preferably surrounding the cut tissue, as best shown in FIGS. 6 (a), 6 (b) and 6 (c). While one skin contacting channel and one suction connector can be utilized, as shown two sets are provided and preferred. Suitable pressure suction source(s) are typically available in surgery rooms. As shown in FIG. 6 (a), skin contacting channel 58 is connected to suction connector 52, and as shown in FIG. 6 (b), skin contacting channel 56 is connected to suction connector 54. Skin contacting channel 58 creates a negative pressure zone, i.e., below atmospheric pressure, with a circumferential seal and tubing connection to gather any leakage of cavity fluid from around the cannula, while skin contacting channel 56 which is located concentrically outside of channel 58 functions to seal and retain cannula 10 to the patient's skin to prevent pull-out and to keep the cannula low profile, i.e., at its shortest possible useable length.

Alternatively, both channels 56 and 58 are connected to a single suction source. One or both channels act as a barrier against the bodily fluid leaked from the body cavity or cut tissue. Inner channel 58 can also remove the leaked bodily fluid that flowed under suction collar 14, and outer channel can maintain the seal with the patient's skin.

Preferably, the negative pressure in inner channel 58 evacuates the leaked bodily fluid and the negative pressure in outer channel 56 retains the suction collar and/or the cannula to the patient's skin, as illustrated in FIG. 6 (c). Alternatively, the functions of channels 56 and 58 can be switched. Furthermore, each channel can perform both functions.

In another embodiment, suction collar 14 comprises a flexing zone 60, which preferably has a sinusoidal or wavy profile, as best shown in FIGS. 1 (b), 6 (a) and 6 (b). Flexing zone 60 has segments of different diameters and allows the proximal portion of cannula 10 to flex. Experiments have shown that cannula 10 can angularly flex up to 15° angle from the centerline of cannula 10. While flex zone 60 can operate without one or both skin contacting channel 56, 58, preferably flex zone 60 cooperates with at least one skin contacting channel 56, 58 or both. Preferably, suction collar 14 has a low vertical profile, i.e., to lie as flush as possible to the patient's skin. FIG. 6 (d) shows an advantage of flexing zone 60 on suction collar 14 when deployed on cannula 10. In certain situations, it is more advantageous to insert cannula 10 at an angle away from the perpendicular axis to the patient's skin, as illustrated. Flexing zone 60 can be compressed on one side, e.g., the right side of FIG. 6 (d), and can be stretched on the other side, e.g., the left side of FIG. 6 (d), while outer channel 56 can provide suction on the skin to maintain cannula 10 in place during the procedure.

As shown herein, suction collar 14 with or without flex zone 60 is provided as an attachable separate component and is clamped or sandwiched between top and bottom flange 16a and 16b of cannula 10. Suction collar 14 can also be attached to any known cannulas in the art to provide retention of the cannulas. Alternatively, suction collar 14 can be made integral to cannula 10 or any known cannulas. Flex zone 60 can also be incorporated into any cannulas known in the art to provide angular flexing.

In another embodiment, suction collar 14 is used in conjunction with a cannula having a fluid or liquid evacuation zone. As illustrated in FIG. 6 (e), a cannula 80 has an inner cannula body 82 that provides structure for the cannula and defines a lumen and an outer concentric sleeve 84, which is porous and preferably is open-cell and non-collapsing. As shown, sleeve 84 is fluidly connected to a suction through a channel, which could be channel 56 or 58 or suction connector 52 or 54, or a combination thereof. Suitable porous materials include, but are not limited to, Porex™ Tubular Micro Filtration (TMF), which is made from polyethylene, polypropylene or ultra-high MW polyethylene. Porous sleeve 84 is positioned adjacent to the cut tissue and in the event of leaked bodily fluid from the cut tissue or leaked insufflated fluid, the leaked fluid could be evacuated by porous sleeve 84 by suction source.

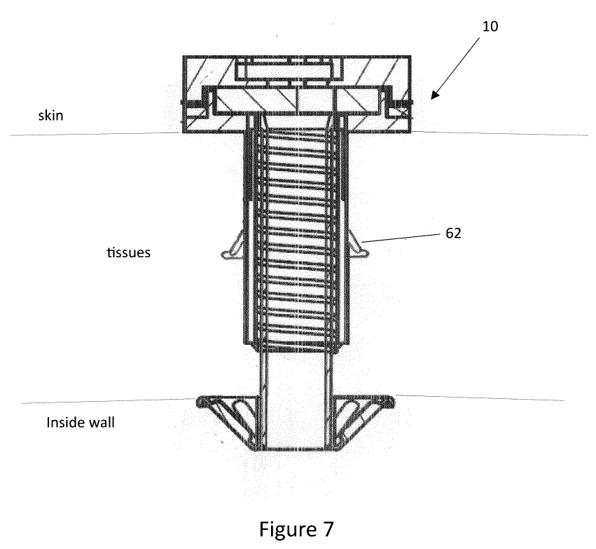

In another embodiment, a grommet, ring, or inverted umbrella, similar to umbrella 22, is added to the outer surface of outer sleeve 18. This grommet, ring or inverted umbrella would press to the cut tissue and would serve as barrier against bodily fluids leaking around the outside of the cannula during surgery. As best shown in FIG. 7, barrier umbrella 62 is positioned on an outside surface of cannula 10 and after deployment would be pressed against the cut tissue. The orientation of umbrella 62, as shown in FIG. 7, is a barrier to bodily fluid leaking around the outside of cannula 10. It is noted that in the reverse orientation, umbrella 62 can also be a barrier to leaking fluid.

In yet another embodiment, a diaphragm or seal is located at the distal end of inner sleeve 20, preferably in addition to the seal provided by proximal diaphragm 24. The distal diaphragm has slits defined thereon to allow in the insertion and withdrawal of surgical instrument. The slits are sized and dimensioned to allow trocar 12 to pass through the diaphragm. The diaphragm would also be a barrier to cavity fluid from leaving the cavity through the lumen or inside channel of cannula 10. As best shown in FIG. 5, distal diaphragm 24A is positioned at the distal end of cannula 10, proximate to umbrella 22. Distal diaphragm 24A is similar to diaphragm 24 located proximally, albeit smaller in dimensions and with slits 26. This diaphragm 24A could be and is preferably made integral to distal anchor or umbrella 22. Distal diaphragm 24A can also be added to the same location in the cannulas shown herein, for example the cannula shown in FIG. 7. U.S. Published Patent Application Nos. US 2022/0110655 and 2023/0037315 disclose an embodiment of a cannula 280 with a proximal seal and a distal seal 296. These two references are incorporated by reference into the present application in their entireties. Alternatively, and less preferably, distal diaphragm 24A may be replaced by a distal flapper valve or a distal duckbill valve.

This embodiment preferably has proximal diaphragm, such as diaphragm 24, distal diaphragm 24A and distal anchor 22. A combination of proximal seal/diaphragm and distal seal/diaphragm limits the amount of liquid that can flow out from the cannula, since during the insertion and/or withdrawal of medical instruments into and out of the cannula at least one seal would be in the sealing position for a longer period. The distal diaphragm could have threads that could be screwed on to inner sleeve 20, or could be attached to inner sleeve 20 via epoxy or other permanent adhesives. Preferably and optionally, a movable suction collar 14 is added to this embodiment to allow the effective length of the cannula in this embodiment to be adjusted, as discussed below.

In an alternative embodiment, cannula 10 is usable without trocar 12. Referring to FIG. 2, barb 42 on inner sleeve 20 may be pulled or pushed down and past channel 44 of outer sleeve 18. From that position, the surgeon may manually rotate the sleeves relative to each other to misalign barb 42 from channel 44, preferably slightly, so that barb 42 rests on the distal end or bottom of outer sleeve 18. In this configuration, cannula 10 is at its maximum length prior to deployment. With its maximum length being temporarily maintained by the misalignment of barb 42 and channel 44 and by barb 42 resting on the distal end of outer sleeve 18, the surgeon may insert the cannula into the cut tissue.

Thereafter, due to the relatively larger diameter of the outer sleeve and also due to the contact between cut tissue and the outer sleeve being potentially longer than that with the inner sleeve, the frictional force on the outer sleeve is higher than that on the inner sleeve. Moreover, it is believed that the inner sleeve is anchored in the inner capsular tissues as the surgeon gently pulls on the cannula; this secures the inner sleeve as the surgeon then twists the cap/outer sleeve to produce relative motion between the inner and outer sleeve. A relative motion or rotation between the inner and outer sleeve toward realignment of barb 42 to channel 44 would rotate the inner sleeve more than the outer sleeve to achieve realignment. Once barb 42 realigns with channel 44, inner sleeve 20 is automatically moved by the spring force stored in spring 28 to automatically readjust the cannula's length, as discussed above. Alternatively, a medical instrument including a trocar can be used to rotate the inner sleeve toward realignment.

In a variation of this embodiment, a distal notch 45 can be placed at the distal end of each channel 44 to receive barb 42. This distal notch would limit the relative rotational movement between inner sleeve 20 and outer sleeve 18.

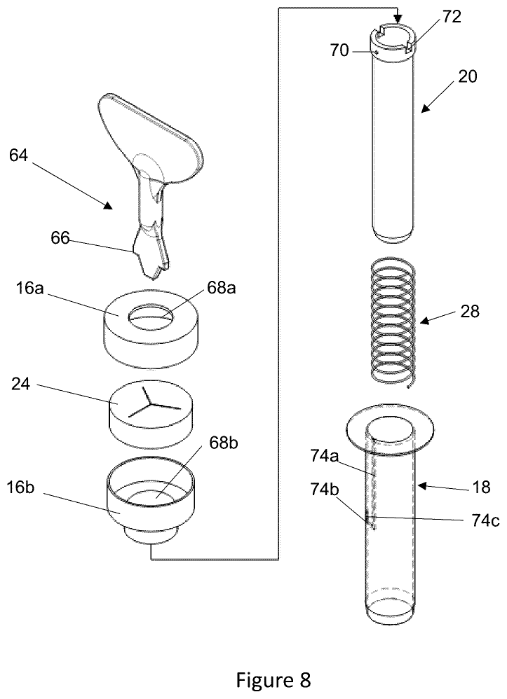

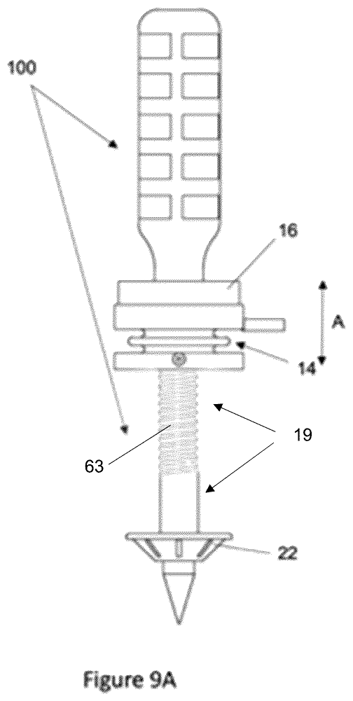

Another variation of this embodiment is shown in FIG. 8. For simplicity, certain elements and features are omitted; only elements and features that are different than the embodiments illustrated in FIG. 2 are discussed and illustrated. As illustrated in FIG. 8, trocar 12 is replaced by key 64, which has at least one shoulder 66. Key 64 is sized and dimensioned to fit through holes 68a and 68b of flanges 16a and 16b, respectively, as well as gasket 24. Shoulder 66 fits into a slot 72 on the proximal end of inner sleeve 20. Inner sleeve 20 further comprises a pin 70 that is adapted to fit into and slide along channels 74a and 74b on outer sleeve 18. Channel 74a orientates along the longitudinal axis of outer sleeve 18, and channel 74b orientates along a circumferential direction. Preferably, channels 74a and 74b are substantially orthogonal to each other.

To insert the cannula of FIG. 8, the surgeon or another medical personnel would pull outer sleeve 18 relative to inner sleeve 20, so that pin 70 rides along channel 74a. Thereafter, the two sleeves are rotated relative to each other, so that pin 20 rides along channel 74b and being held in channel 74b by the force of spring 28. Optionally, a short longitudinal channel 74c is provided orthogonally to channel 74b, as shown, to securely lock pin 70 and to maintain the cannula in the extended configuration for insertion. After insertion, key 64 is inserted into holes 68a and 68b and shoulders 66 is received into slots 72 to rotate inner sleeve 20 in the opposite direction to realign pin 70 with channel 74a to allow spring 28 to automatically readjust the length of the cannula, as discussed above. Key 64 is also capable of pushing inner sleeve 20 longitudinally a distance sufficient to release pin from channel 74c, if present.

The present inventors have conducted experiments and provided examples of the inventive cannulas with telescoping cannula body, which are discussed in the Appendix.

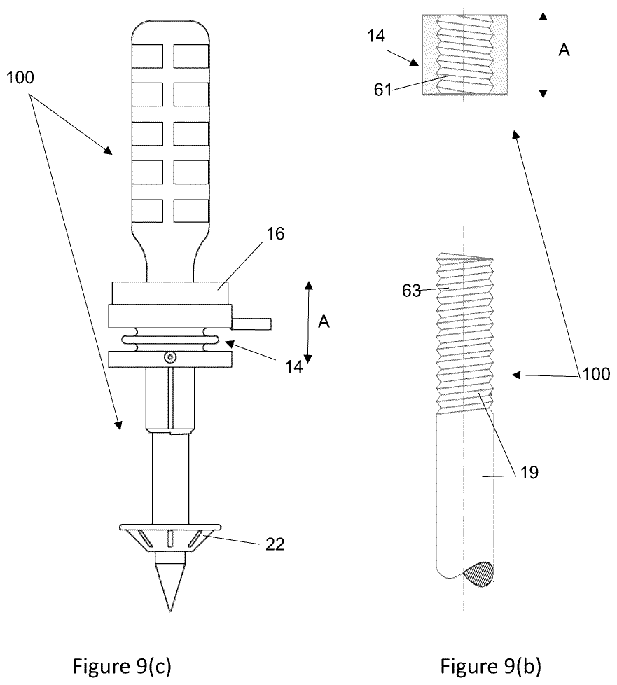

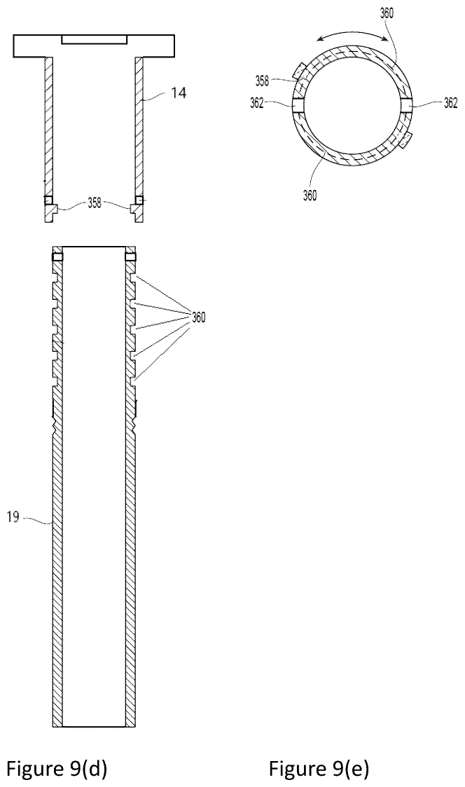

Other embodiments of the present invention, surgical cannula 100, are shown in FIGS. 9A-9B and 9C. In these embodiments, the length of cannula may be fixed or unchanged, i.e., the cannula body does not need to be telescopic to adjust the cannula's effective length, and the movable suction collar 14 is moved to contact the skin after cannula 10 is inserted into the body cavity.

Referring to FIGS. 9 (a) and (b), suction collar 14 is movable along the longitudinal length of cannula 100 along direction A. The cannula body preferably has single-piece cannula body 19, as illustrated. After insertion into the body cavity, a distal retainer 22, which can be an inverted umbrella or a distal balloon, is deployed, cannula 100 is slightly withdrawn until distal retainer 22 engages with the cavity wall. Suction collar 14 is moved distally until it is flushed with the skin to adjust the cannula's effective length to match the thickness of the cut tissue. Suction is then activated, as discussed above, to retain the suction collar in place and to evacuate bodily liquid. Flange 16 is preferably stationary or may move along with suction collar 14. Flange 16 can be of smaller diameter than suction collar 14 and may approach the diameter of cannula body 19. Suction collar 14 may or may not contain flexible section. Alternatively, distal retainer 22 is omitted. The surgeon would know the depth of the cut tissue and would insert cannula 100 through the cut tissue and lower suction collar 14 until it is flushed with the skin. A trocar, such as trocar 12 described above, can hold the cannula in place. Suction is then activated to retain cannula 100 in place.

In the embodiment of FIGS. 9 (a) and (b), suction cup 14 is moved by rotation. Referring to FIG. 9 (b), suction collar 14, shown schematically with cannula body 19, is rotationally movable along the longitudinal length of the cannula body. Suction collar 14 has internal threads 61, which are sized and dimensioned to fit over external threads 63 on cannula body 19. After cannula 100 is inserted into the body cavity, as discussed in the preceding paragraphs, suction collar is rotated in one direction, preferably clockwise, to lower the suction collar until it contacts the skin. The suction source is then connected at suction connector 52, 54 and suction activated.

Alternatively, the cannula body may comprise outer sleeve 18 and inner sleeve 20, as shown in FIG. 9 (c) or may have a fixed length, such as cannula body 19 but without the threads. Preferably, the frictional contact between suction collar 14 and the outer surface of the cannula body is sufficient to prevent relative movement between the suction collar 14 and the cannula body in the absence of force applied by the surgeon. There may also be an O-ring along the inner diameter of the suction collar to further seal it to the cannula and provide additional friction fit.

In another embodiment, suction collar 14 and cannula body 19 have a bayonet-type connection, as shown in FIGS. 27E and 27F of U.S. published patent application No. US 2022/0110655 to Mikol et al., which is incorporated herein in its entirety. These figures are partially incorporated herein as FIGS. 9 (d) and 9 (e). In this alternative, suction collar 14 has at least one or a pair of inward projections (358) and cannula body 19 has a series of locking channels (360) and lateral sliding channels (362) formed on its outer surface. Locking channels (360) and sliding channels (362) are sized and dimensioned to receive projections (358). When projections (358) are aligned and received in sliding channels (362), suction collar 14 can slide up and down cannula body 19. When suction collar 14 contacts the skin surrounding the cut tissue, projections (358) can slide into the closest locking channels (360) to retain the suction collar 14 in place. It is noted that projections (358) can be positioned on cannula body 19, which would be outward facing, and locking and sliding channels can be positioned on the inside surface of suction collar 14. It is noted that reference numbers that are in parenthesis in this and following paragraphs are from US 2022/0110655.

To remove cannula 10 after the completion of the surgical procedure, suction collar 14 can be counter rotated in the embodiment of FIGS. 9 (a) and (b), or can be slid proximally in the embodiment of FIG. 9C before the cannula is withdrawn as discussed above. In the embodiment with the bayonet connection, projections (358) are moved to sliding channels (362) and suction cup 14 is then moved proximally.

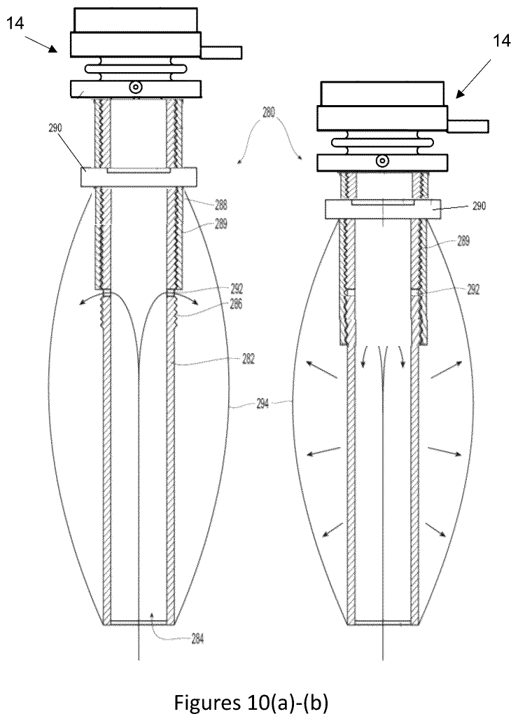

In yet another embodiment of the present invention, an embodiment of U.S. Published Patent Application Nos. US 2022/0110655 and 2023/0037315, which are incorporated by reference, is improved. A telescopic cannula (280), i.e., having a cannula body with an inner member (282) and an outer member (289), shown in FIGS. 24A-B and 25A-B of the '655 and '315, has a suction collar 14 added to the proximal end, as shown in FIGS. 10A-B of the present application. Preferably suction collar 14 is movable, as described above. Relevant excerpts from the description from the '655 and '315 applications are reproduced below.

A simplified cannula (280) is shown in FIGS. 10 (a) and (b). Cannula (280) has at least one casing (282) defining lumen (284). Casing (282) has outer threaded connection (286) sized and dimensioned to connect to inner threaded connection (288) on downward skirt (289) of cap (290). Casing (282) has port or channel (292), which is preferably located proximate to the top thereof. After cannula (280) is inserted into the surgical site, the insufflated liquid enters the distal end of casing (282) under pressure and inflates outer membrane (294). After outer membrane (292) is filled, cap (290) is rotated further downward to cover and seal port (292) to isolate the insufflated liquid inside outer membrane (294) and to compress outer membrane (294) to increase its internal pressure and width to ensure a more secure placement at the surgical site. Pressure is increased by skirt (289) being pushed into the internal space of outer membrane (294) and/or by the top of cap (290) pushing down on outer membrane (294). Preferably, outer membrane is attached to cannula (280) independent of cap (290), i.e., not attached to cap (290) or skirt (289), so that as cap (290)/skirt (289) rotates outer membrane (294) is not twisted.

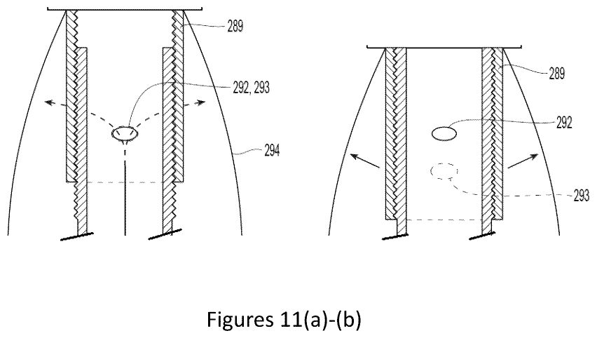

Another version of cannula (280) is illustrated in FIGS. 11 (a) and (b). In this version, skirt (289) of cap (290) has port (293). When port (293) aligns with port (292) of casing (282), as shown in FIG. 11 (a), insufflated fluid can enter lumen (284) and go through ports (292) and (293) to inflate outer membrane (294). When cap (290) advances distally, ports (292) and (293) misalign, as shown in FIG. 11 (b) and close the fluid communication between lumen (284) and outer membrane (294) and the fluid in outer membrane (294) is compressed, as discussed above.

Suction collar 14, which is preferably movable in the distal direction, is attached to the proximal end of the cannula to provide suction to retain the cannula to the patient's skin and/or to evacuate leaked fluid, as described herein.

Preferably, suction collar 14 replaces cap (290) and skirt/outer member is attached to suction collar 14. Preferably, suction collar 14 and cap (290) are integral with each other, and is collectively referred as cap. Suction collar 14 may or may not have flex section 60 in this embodiment.

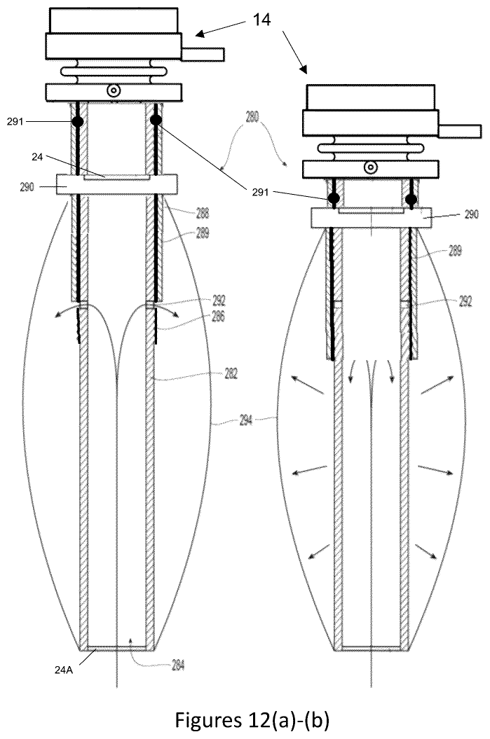

As shown FIGS. 10 (a) and (b) and 11 (a) and (b), members (282) and (289) are rotated by threads to move them relative to each other. Alternatively, these members can slide relative to each other, as described in FIGS. 12 (a) and (b). As shown, the corresponding threads are replaced by smooth inner surface on member (289) and smooth outer surface on member (282), so that these two members are slidable in the proximal-distal directions. Preferably, at least one O-ring 291 is placed between members (282) and (289) to provide a seal therebetween.

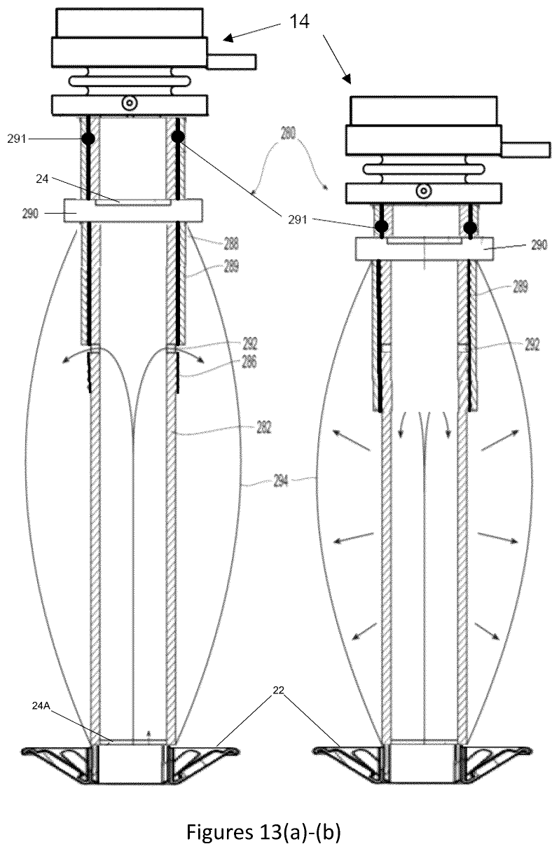

As illustrated in FIGS. 13 (a) and (b), the cannula shown in FIGS. 10 (a) and (b), 11 (a) and (b) and 12 (a) and (b) may have an anchor 22, as described herein, to maintain the cannula in place during the medical procedure. Alternatively, in the embodiment shown in FIGS. 13 (a) and (b), outer membrane (294) can be omitted, as well as ports (292), (293). Maintaining the cannula in place is accomplished by anchor 22 and suction collar 14, as described herein.

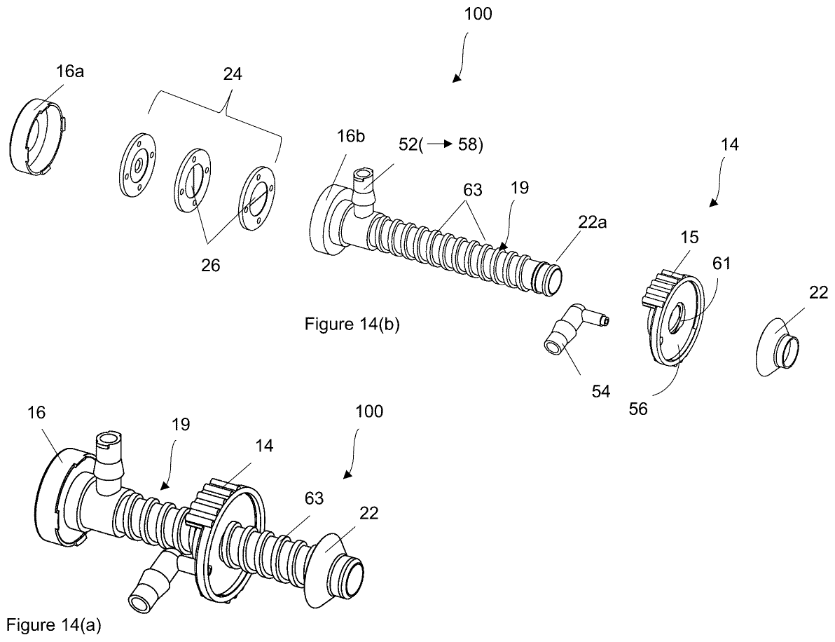

Additional embodiments of the cannulas with a movable suction collar 14 are shown below in FIGS. 14-17 and their subparts. For brevity, components that are similar to the embodiments in FIGS. 14-17 are described only once in connection with FIGS. 14 (a)-(b).

Another version of cannula 100 shown in FIG. 9 (a) and (b) is illustrated in FIGS. 14 (a) and (b). Suction collar 14 has suction connector 54 attached thereto, which is fluidly connected to skin contacting channel 56. In this embodiment skin contacting channel 56 is located on the underside or distal side of collar 14. Cannula body 19 has outer threads 63 which are sized and dimensioned for inner threads 61 of collar 14 to rotate thereon to move collar 13 proximally and distally or up and down relative to cannula body 19. Collar 14 preferably has handgrip 15 to allow the surgeon to readily rotate the collar. Suction connector 52 is this embodiment is connected to cap 16 and may be connected to porous layer 84, as shown in FIG. 6E, which forms a part of cannula body 19 to evacuate bodily fluids from the cut tissue. Cap 16, as shown, comprises top portion 16a and bottom portion 16b and contain gasket 24 with slit 26. As shown gasket 24 comprises two members, each with a slit 26 positioned at an angle from each other. An optional additional gasket can also be included in cap 16, as shown. Anchor 22, which in this embodiment is an inverted umbrella, is snap-fitted over ridges 22a located a distal end of cannula body 19.

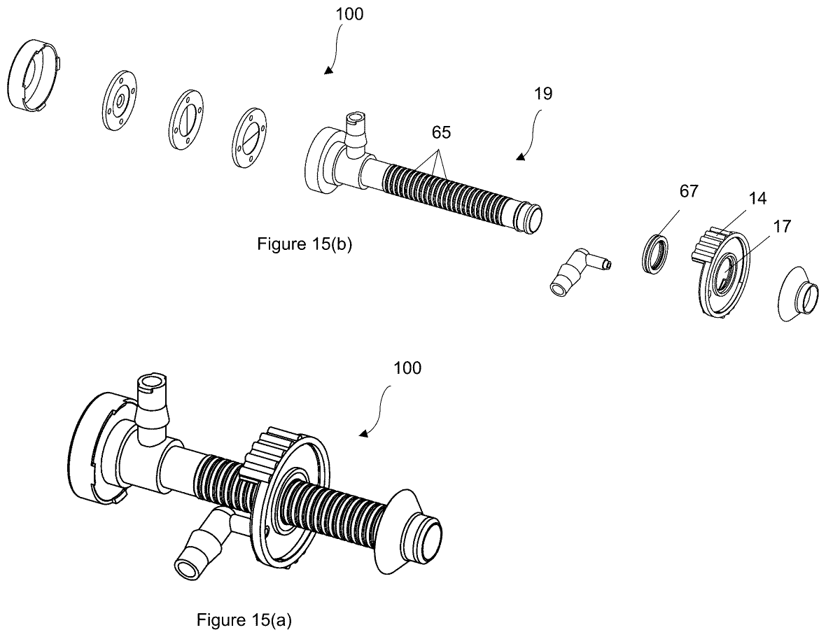

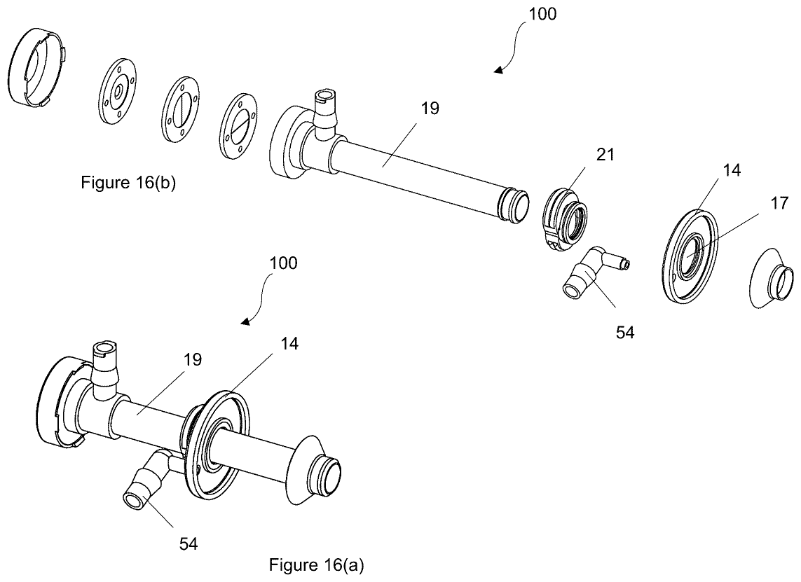

FIGS. 15 (a) and (b) illustrate another version of the cannula with a movable suction collar. Instead of outer threads 63 and inner threads 61, cannula body 19 has circumferential ribs 65 positioned on its outside surface. Suction collar 14 has seal internal ribs 67 fixedly attached to its inner opening 17, for example by threads or adhesion. Internal ribs 67 are sized and dimensioned to fit between two adjacent circumferential ribs 65 and provide a seal therewith. Collar 14 can be moved along cannula body 19 by moving the collar over circumferential ribs 65 using slight force. Such movement advantageously produces a “clicking” sound as the internal ribs 67 of collar 14 are moved over each circumferential cannula rib 65. It is noted that the circumferential ribs and internal ribs are not threads.

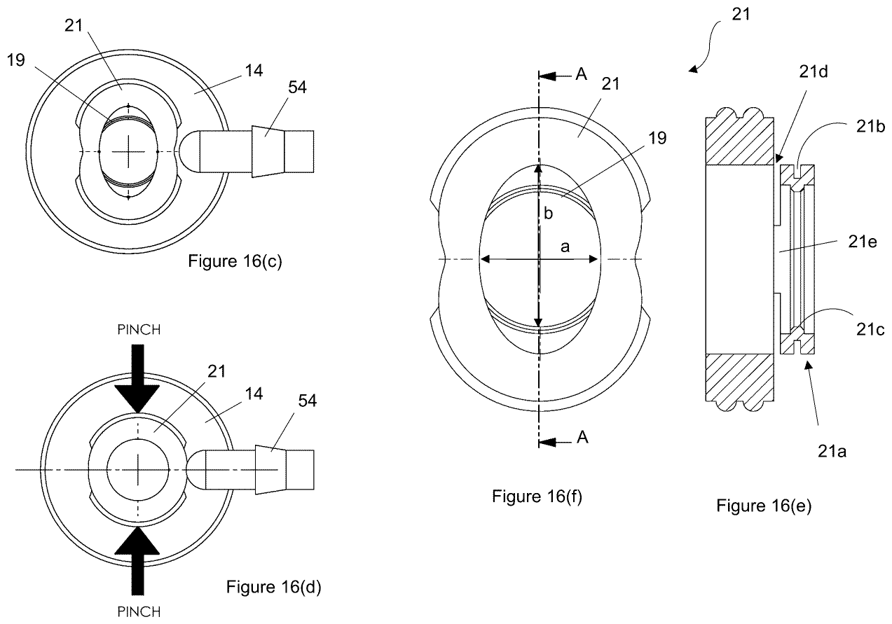

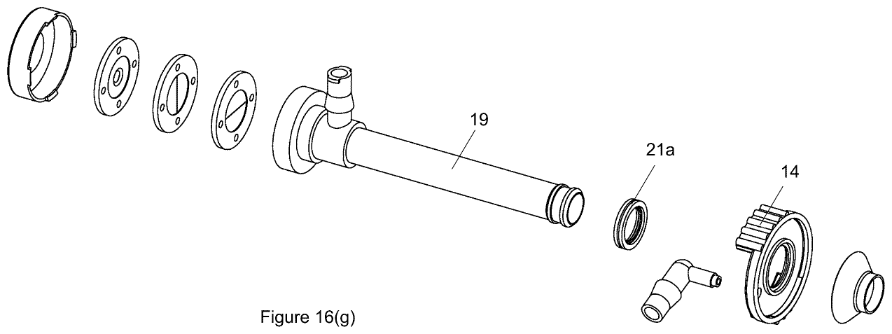

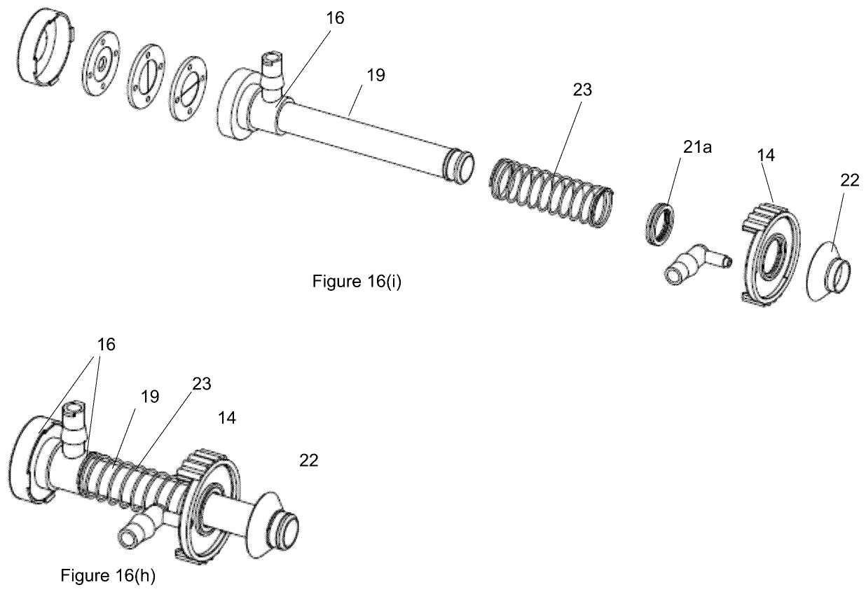

FIGS. 16 (a) and (b) illustrate a cannula that is similar to the cannula shown in FIG. 9 (c). In this embodiment, suction collar 14 moves along cannula body 19, which preferably is not telescopic, using friction contact between suctional collar 14 and cannula body 19. Deformable friction seal 21 is fixedly attached to inner opening 17 of suction collar 14. Deformable friction seal 21 is sized and dimensioned to slide over the outer surface of cannula body 19 and preferably maintain a seal therewith. Referring to FIGS. 16 (c) and (f), deformable friction seal 21 has an oval or elliptical shape with an oval central opening with minor axis “a” and major axis “b”, wherein axis “b” is longer than axis “a”. Minor axis “a” is smaller than a diameter of cannula body 19, so that in a locking position shown in FIG. 16 (c) deformable friction seal 21 grabs onto the cannula body to hold suction collar 14 fixedly in place. To move suction collar 14 along cannula body 19, deformable friction seal 21 is squeezed or pinched in opposite directions along its major axis “b” as illustrated in FIG. 16 (d) so that the central opening of deformable friction seal 21 becomes rounded and larger than the outer diameter of cannula body 19 to allow deformable friction seal 21 to move along cannula body 19. Once the pinching force is released, deformable friction seal 21 resumes the locking configuration shown in FIG. 16 (c).

Referring to FIG. 16 (e), deformable friction seal 21 comprises a connection seal portion 21a to connect to suction collar 14. Preferably, connection seal 21a has cap interface gap 21b sized and dimensioned to receive a corresponding inner ridge in central opening 17 of suction collar 14 to connect deformable frictional seal 21 to suction collar 14. Connection seal 21a also has seal surface 21c, wherein cannula body 19 is received through connection portion 21a and seal surface 21c contacts and seals the outer surface of cannula body 19. Connection seal 21a is separated from the squeezable body of deformable friction seal 21 by gap 21d, so that deformable friction seal 21 can be readily pinched. Connection seal 21a is connected the squeezable body by one or more peripheral segments 21e.

In the embodiment of FIGS. 16 (a)-(f), deformable friction seal 21 of the suction collar 14 or suctioning device comprises a squeezable/deformable locking device connected to the connection seal portion 21a, wherein in a locking configuration the deformable locking device is in a relaxed state and applies a pressure on the cannula body to resist a movement of the suction collar along the cannula body, and wherein in a release configuration the deformable locking device is elastically deformed to allow the movement of the suction collar along the cannula body. Preferably, in the locking configuration the deformable locking device has an oval shape with first portions on its minor axis applying pressure on the cannula body, and in the release configuration second portions on its major axis are elastically deformed to release the first portions, thereby allowing the suction collar to move. Upon release of the pressure, the deformable locking device returns to the locking configuration.

Alternatively, as illustrated in FIG. 16 (g), only connection seal 21a with seal surface 21c is used without the oval or elliptical squeezable portion. Connection seal 21a would fit inside the central opening of suction collar 14. In one variation, connection seal 21a may be structurally the same as internal ribs 67 shown in FIG. 15 (b).

Another version of the cannula is illustrated in FIGS. 16 (h) and 16 (i). This version is the same as that shown in FIG. 16 (g), except that a spring 23 is provided between the bottom of cap 16 and the top of suction collar 14. When the cannula is assembled as shown in FIG. 16 (h) and inserted into a cavity in the patient, spring 23 exerts a force pushing suction collar 14 toward the patient's skin, and the cannula is retained between collar 14 and distal anchor 22. Spring 23 is sized and dimensioned to apply a sufficient force on the skin, i.e., the length of the spring and the spring constant are selected to provide the sufficient force to retain the cannula in place during the medical procedure but not to pull the cannula outward or not to unseat the distal anchor 22 in the cavity. While preferred, the suctioning force emitted from suction collar 14 may be omitted from this embodiment.

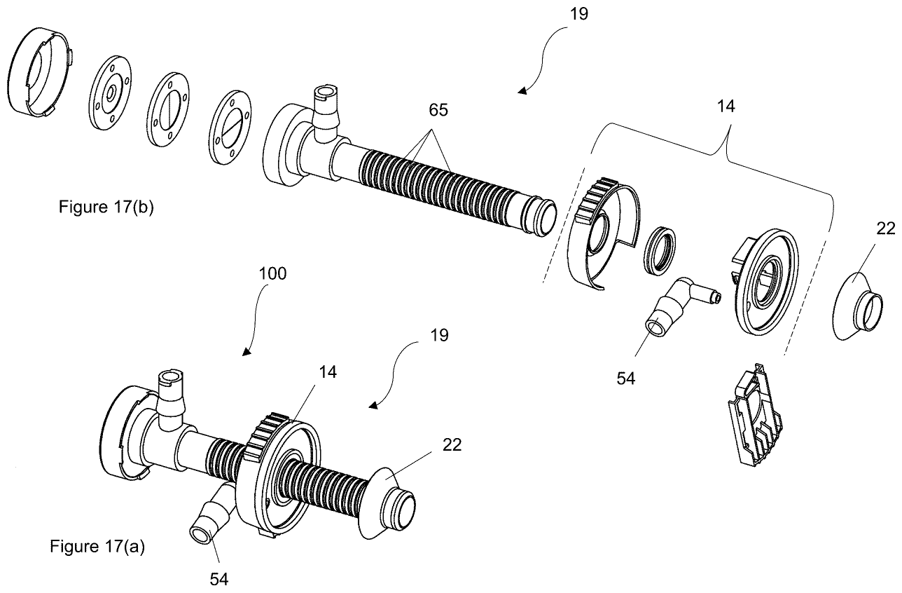

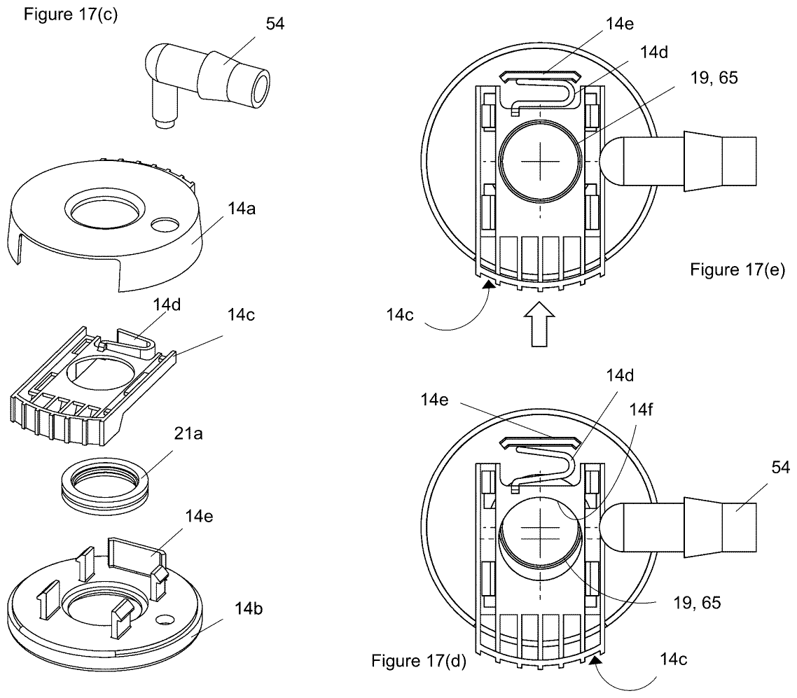

Another cannula embodiment with circumferential ridges 65 disposed on the outer surface of cannula body 19 is shown in FIGS. 17 (a) and (e). Instead of internal ribs 67 on suction collar 14 shown in FIGS. 15 (a) and (b), suction collar 14 in this embodiment comprises an assembly that is configured to switch from a locking configuration to a release configuration. As best shown in FIG. 17 (c), disposed between collar top 14a and collar bottom 14b are a connection seal, which can be the same as connection seal 21a in the embodiment shown in FIGS. 16 (a) and (g), and a biased actuator 14c. Collar top 14a has an opening for actuator 14c to extend out of. Collar bottom 14b has four snap features to connect to collar top 14a. Connection seal 21a is retained in the opening on collar bottom 14b in the same manner discussed in connection with FIGS. 16 (a)-(g). Biased actuator 14c has a spring 14d, which after assembly pushes against stop 14e disposed on collar bottom 14b. Cannula body 19 passes through the central opening in collar top 14a, the central opening in biased actuator 14c, connection seal 21a, which is fixedly connected to the central opening in collar bottom 14b. While spring 14d is illustrated as a leaf spring, spring 14d can be any spring or biasing element, such as a helical spring.

As best shown in FIG. 17 (d), when suction collar 14 is assembled and in the locking configuration, actuator 14c is biased outward by spring 14d, portion 14f of the central opening in actuator 14c engages the space between adjacent circumferential ridges 65 and prevents suction collar 14 from moving along cannula body 19. In the release configuration as best shown in FIG. 17 (e), biased actuator 14c is pushed inward and the central opening of biased actuator 14e aligns with cannula 19 and circumferential ridges 65 freeing suction collar 14 to move along the outer surface of cannula body 19. In other words, portion 14f is moved away from circumferential ridges 65. Once biased actuator 14c is released, suction collar 14 returns to the locking configuration of FIG. 17 (d).

While it is apparent that the illustrative embodiments of the invention disclosed herein fulfill the objectives stated above, it is appreciated that numerous modifications and other embodiments may be devised by those skilled in the art. One such modification is that spring 28 could be extended in the configuration of FIG. 4. In this modification spring 28 is attached at each end to ledge 46 and 48, respectively, so that when trocar 12 pushes into cannula 10's lumen spring 28 is extended to store energy. Therefore, it will be understood that the appended claims are intended to cover all such modifications and embodiments, which would come within the spirit and scope of the present invention.

### APPENDIX

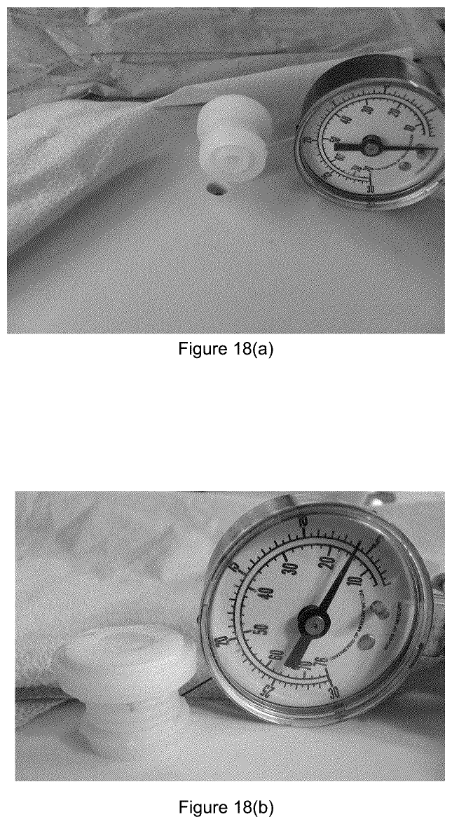

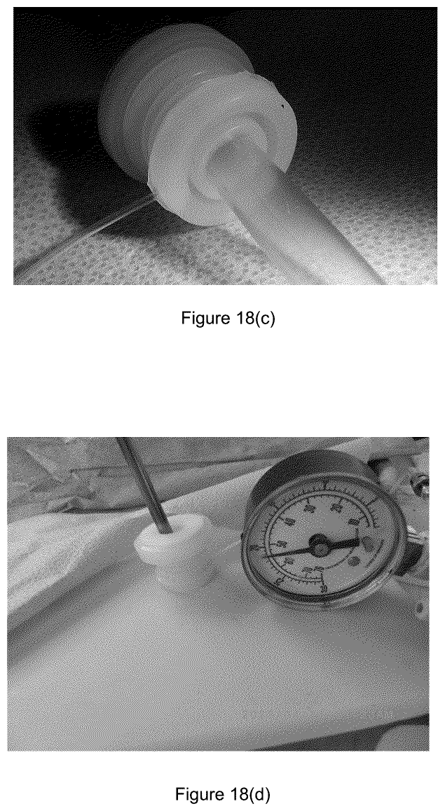

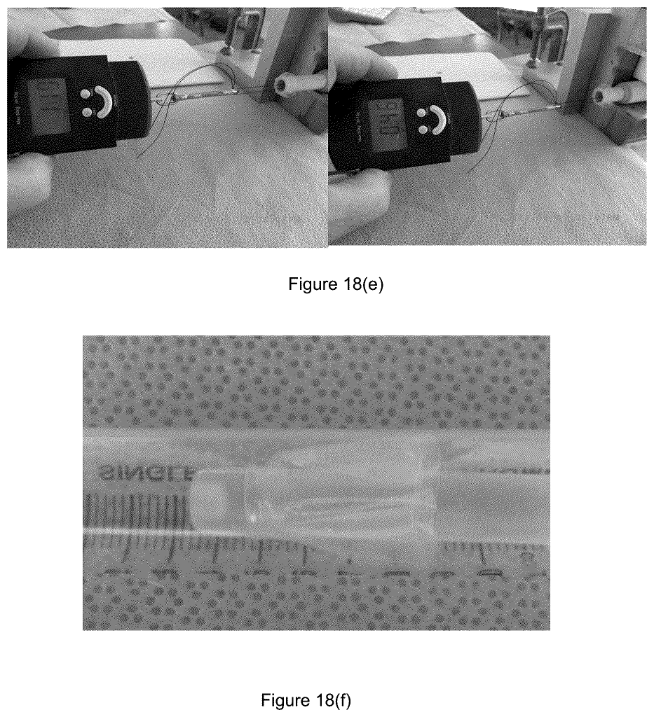

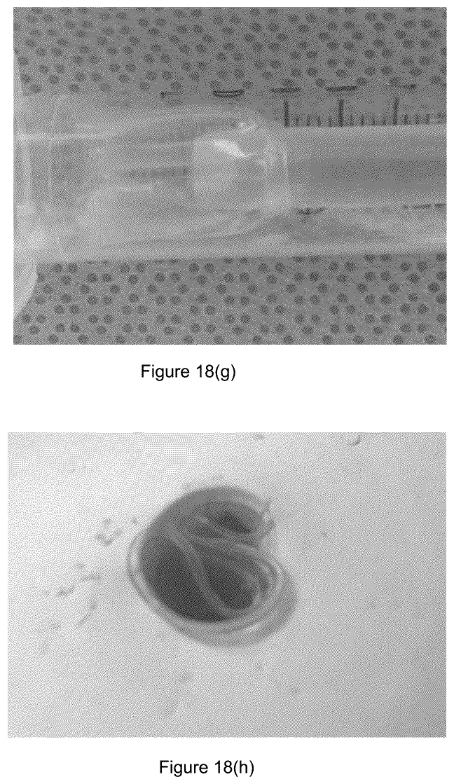

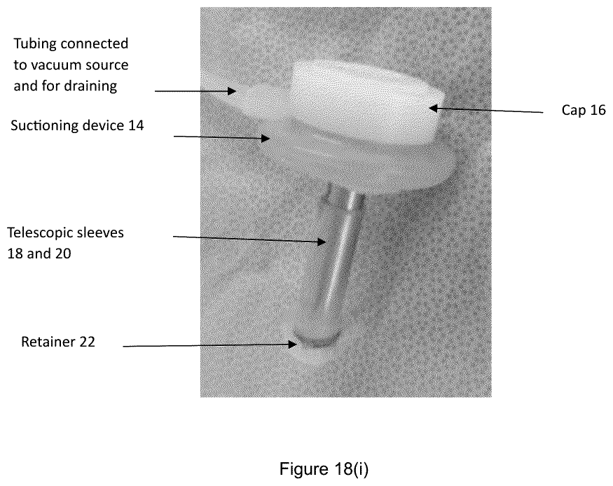

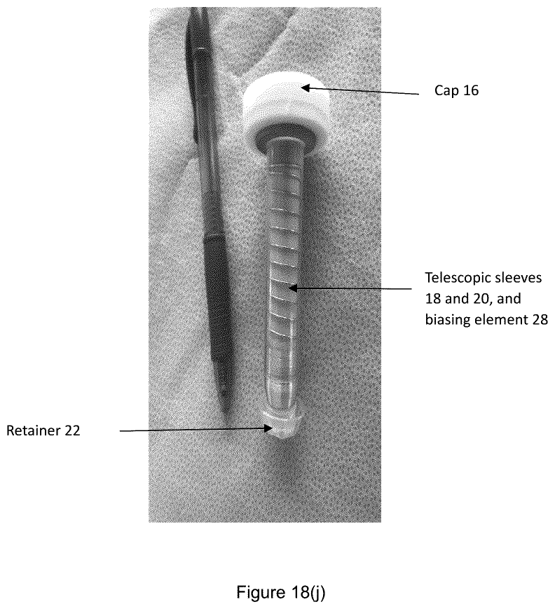

Experiment 1 shows that suction collar 14 can maintain the location of cannula 10 relative to the patient's skin. FIG. 18 (a) is a photograph showing suction collar 14 before being connected to the suction source with the pressure gage reading at zero. The suction collar is connected to the suction source when the inner lumen of the suction collar is connected to the hole shown. The suction force is generated in the circular channel pressed against the surface surrounding the hole. FIG. 18 (b) is a photograph showing the static (without simulated operative tool movement force inputs) suction stabilization to a dry skin surrogate polymer sheet was achieved with a proof of concept molded silicone model. Stabilization occurred at negative pressure of ˜6 psi (−310 mm Hg) with this concept design acting on annular suction area of 0.147 in². This equates to a force of 0.9 pounds. Because the typical tissue contact surgical stabilizers based on negative pressure operate at a limit of −250 mm Hg, the annular suction area would need to increase to reduce the operating pressure. If the annular area was increased to 0.185 in², an operating suction limit of −250 mm Hg would achieve 0.9 pounds of force. Actual clinical requirements of force will determine the optimum suction area.

FIG. 18 (c) is a photograph showing the skin sealing geometry and annular suction area of the proof of concept silicone model in Photo 2. The sealing geometry is shown to be concentric and circular. Actual clinical requirements may utilize non-concentric, curvilinear and variable width/shape sealing areas. FIG. 18 (d) is a photograph showing that this pressure/force is sufficient to support a surgical instrument, represented by a metal rod of similar weight and shape.

Experiment 2 shows a simulation of the available force from a clinically relevant sized low profile wire wound spring operating to retract the distal cannula section into the proximal cannula section. The experiment does not include an approximation of final design frictional losses of injection molded components nor the final design spring. FIG. 18(e) are photographs showing forces measured were ˜1.1 pounds at full extension to 0.45 pounds at full retraction (shortened length).

Experiment 3 shows the shape and orientation of the umbrella anchor during a mockup insertion and withdrawal of the cannula. FIG. 18 (f) is a photograph showing the orientation of umbrella 22 as it advances with cannula 10 into the patient's cavity (right-to-left direction). FIG. 18 (g) is a photograph showing umbrella 22 during the withdrawal of the cannula (left-to-right direction). FIG. 18 (h) is a photograph showing a withdrawal of the cannula and umbrella through simulated tissue.

Example 4. FIG. 18 (i) is an annotated photograph showing a prototype of a version of inventive cannula 10.

Example 5 shows non-limiting, exemplary dimensions and properties of biasing element 28. As used herein, a compression spring is the cylindrical coiled mechanical device which stores and releases energy to automatically to shorten the cannula. This spring is preferably an open-coil helix wound or constructed to oppose compression along the axis of wind. The wire used to manufacture this helical spring is tempered to create optimum compression characteristics. This spring is assembled over a guide tube (distal cannula section) and fitted inside a bore (proximal cannula section). Specifications of the compression spring may be varied to deliver the desired force to shorten the cannula. For example, non-limiting, exemplary manufacturing specifications for creating three distinct compression forces (spring rate) are listed below. These examples demonstrate the range and general description of the compression spring features which will deliver self-shortening clinical performance.

### Example 5.A

Spring rate: 1.05 lbs./in

Wire diameter: 0.025, 302 stainless steel

Outer diameter: 0.430

Inner diameter: 0.380

Free length: 2.0″

Active turns: 7

Total turns: 9

Solid height (fully compressed): 0.2125″

Load @ 1.0″ compression: 1.05 lbs.

Load near full compression: 1.869 lbs.

Ends: square & ground

### Example 5.B

Spring rate: 2.10 lbs./in

Wire diameter: 0.032, 302 stainless steel

Outer diameter: 0.444

Inner diameter: 0.380

Free length: 2.0″

Active turns: 8.92

Total turns: 10.92

Solid height (fully compressed) 0.3336″

Load @ 1.0″: 2.10 lbs.

Load near full compression: 3.465 lbs.

Ends: square & ground

### Example 5.C

Spring rate: 3.15 lbs./in

Wire diameter: 0.036, 302 stainless steel

Outer diameter: 0.452

Inner diameter: 0.380

Free length: 2.0″

Active turns: 9.26

Total turns: 11.26

Solid height (fully compressed): 0.3873

Load @ 1.0″ compression: 3.15 lbs.

Load near full compression: 4.977 lbs.

Ends: square & ground

Example 6. FIG. 18 (j) is an annotated photograph showing another prototype of a version of inventive cannula 10 shown with a pen for scale.

We claim:

1. A suctioning device adapted to be attached to a cannula, said suctioning device comprises at least one skin contacting channel fluidly connected to a suction source, wherein the at least one skin contacting channel provides a suction force.

2. The suctioning device of claim 1, wherein the at least one skin contacting channel provides the suction force to retain the cannula to the patient's skin.

3. The suctioning device of claim 1, wherein the at least one skin contacting channel provides a suction force to evacuate fluid leaked around the cannula.

4. The suctioning device of claim 1 is positioned below a proximal flange on the cannula.

5. The suctioning device of claim 1, wherein the suctioning device is movable relative to a body of the cannula.

6. The suctioning device of claim 1 further comprising a flex section allowing a proximal section of the cannula to flex about its centerline.

7. The suctioning device of claim 1, wherein the at least one skin contacting channel comprises a first skin contacting channel to retain the cannula to the patient's skin, and a second skin contacting channel to evacuate fluid leaked around the cannula.

8. The suctioning device of claim 7, wherein the first and second skin contacting channels are concentric and are connected said suction source.

9. The suctioning device of claim 1, wherein the at least one skin contacting channel is connected to a porous, open-cell sleeve on the cannula.

10. A cannula comprising
a cannula body,
an optional distal anchor disposed on a distal end of the cannula body, and
the suctioning device according to claim 1 attached to an outside surface of the cannula body, wherein the suction force provided the suctioning device retains the cannula to a patient's skin,
wherein the suctioning device is movable relative to the cannula body.

11. The cannula of claim 10, wherein the suctioning device is slidable along the cannula body.

12. The cannula of claim 11, wherein the suctioning device is connected to the cannula body through a bayonet type connection.

13. The cannula of claim 11, wherein the suctioning device is connected to the cannula body by a frictional seal.

14. The cannula of claim 13, wherein the suctioning device comprises a squeezable locking device connected to the frictional seal, wherein in a locking configuration the squeezable locking device is in a relaxed state and applies a pressure on the cannula body to resist a movement of the suctioning device along the cannula body, and wherein in a release configuration the squeezable locking device is elastically deformed to allow the movement of the suctioning device along the cannula body.

15. The cannula of claim 14, wherein in the locking configuration the squeezable locking device has an oval shape with first portions on its minor axis applying pressure on the cannula body, and wherein in the release configuration second portions on its major axis are elastically deformed to release the first portions.

16. The cannula of claim 11, wherein the suctioning device is connected to the cannula body by a frictional seal, and the suctioning device is biased toward the patient's skin by a spring, and wherein the distal anchor is present.

17. The cannula of claim 11, wherein the cannula body comprises a plurality of circumferential ridges on its outer surface and the suctioning device comprises at least one inner ridge, wherein the at least one inner ridge is received between adjacent circumferential ridges.

18. The cannula of claim 11, wherein the cannula body comprises a plurality of circumferential ridges on its outer surface and the suctioning device comprises a biased actuator comprising at least one locking edge, wherein in a locking configuration the at least one locking edge is received between adjacent circumferential ridges and wherein in a release configuration the at least one locking edge is moved away from the plurality of circumferential ridges.

19. The cannula of claim 18, wherein the suctioning device is moved from the locking configuration to the release configuration by pushing an actuating element on the biased actuator.

20. The cannula of claim 10, wherein the suctioning device is rotatable along the outside surface of the cannula body.

21. The cannula of claim 20, wherein the suctioning device is connected to the cannula body through threads.

22. The cannula of claim 10, wherein the suctioning device can be positioned at an adjustable position along a length of the cannula body.

23. The cannula of claim 10, wherein the cannula body comprises a telescopic body or a single-piece body.

24. A cannula comprising:
a casing defining a lumen sized and dimensioned to receive one or more medical instruments,
an inflatable outer membrane attached to an outer surface of the casing, and
a cap sized and dimensioned to move relative to the casing,
at least one port formed on the casing, wherein the at least one port is fluidly connecting the outer membrane to the lumen,
wherein the outer membrane is filled with a fluid and the cap is moved in a distal direction to fluidically isolate said at least one port from the outer membrane to further pressurize the outer membrane
wherein the cap comprises the suctioning device according to claim 1, wherein the suction force provided the suctioning device retains the cannula to a patient's skin.

25. The cannula of claim 24, wherein the cap comprises a second port and wherein the second port and said at least one port are aligned to fluidly connect the outer membrane to the lumen.

26. The cannula of claim 24, wherein the cap is threadedly connected to the casing so that relative rotation between the cap and the casing allows the cap and the casing to move relative to each other.
